(19) United States
(12) Patent Application Publication
(10) Pub. No.: US 2006/0003314 A1
(43) Pub. Date: Jan. 5, 2006

(54) Methods for peptide and protein display in nucleic acid arrays

(76) Inventor: Bjorn H Lindqvist, Oslo (NO)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/507,434

(22) PCT Filed: Mar. 13, 2003
(86) PCT No.: PCT/NO03/00088

§ 371 Date: Aug. 24, 2005

(30) Foreign Application Priority Data
Mar. 15, 2002 (NO) 20021298

(51) Int. Cl. C12Q 1/70; C12Q 1/68; C12N 15/74

(52) U.S. Cl. 435/5; 435/6; 435/472

(57) ABSTRACT

Virus displayed peptides or proteins can be integrated and presented in a nucleic acid array format. The present invention makes it possible to generate naked nucleic acidvirion protein display complexes in which the individual nucleic acid template is freely and covalently linked to the very same virion proteins it coded for. This cis-capture of the virion proteins by its naked nucleic acid template makes it possible to use phage display libraries in combinatorial display array formats. This can be achieved by allowing the naked nucleic acid-virion protein display complexes to be deposited by nucleic acid hybridisation to their corresponding mRNA or cDNA which separately have been prepared and used for the fabrication of a nucleic acid array. The hybridisation step will therefore function as a ‘search engine’ and a ‘delivery robot’ by automatically positioning the protein to its own gene. The nucleic acid coded protein or peptide display arrays described here can be used in functional genomics, protenomics and in protein or peptide identfication of relevance for the exploration of therapeutic drugs as well as for search of new diagnostic procedures.

### FIELD OF THE INVENTION

The present invention relates to a display virion and methods of creating a nucleic acid based protein display array, where a diverse population of peptides or proteins is displayed and the use thereof.

### BACKGROUND OF THE INVENTION

Recent advances in array technology have led to high through-put analysis of gene expression at the level of transcription. To achieve functional array based genomics, however, the proteins encoded by the genes in the arrays should also be present and displayed and presented (Cahill, 2000, Irving and Hudson, 2000).). Although it is expected that a well-defined protein array would be a powerful tool in better understanding the mechanisms in a cell, organ and organism, it has proven very difficult to reproducibly manufacture large protein microarrays (Mitchell, 2002). Many different approaches have been considered and tested, without being able to duplicate the easy and power of the more established DNA microarray technology. The present invention overcome these challenges by utilising the already established DNA microarray technology in a method where the protein encoded by the gene is indeed present, displayed and presented. This has been made possible by taking advantage of a published observation (Chattoraj and Inman, 1974, Thomas, 1974) that certain virus are able to release their chromosome from their capsids while remaining covalently attached to their tail or head and combine this observation with phage display and nucleic acid array technologies. By inserting an exogenous nucleic acid encoding a product into the virus genome, it is possible to achieve a covalent attachment between the exogenous nucleic acid and its encoded product. The present invention provides thereby a new and more simple method in creating a nucleic acid based protein display array.

Nucleic acid array primarily DNA arrays, may contain thousands of individual nucleic acid addressable entries on a small surface (as small as one square cm) fabricated by robotic procedures involving fine mechanics and special chemistries. Phage display is a process by which a peptide or a protein is expressed—almost invariably as an exterior fusion to the surface protein of the phage particle in such a way that the peptide or protein sequence can be deduced from the coding DNA or RNA sequence residing in the phage particle or its transductant.

U.S. Pat. No. 6,207,446 describes methods for selection of protein molecules that make use of RNA-protein fusions, where a microchip comprises an array of immobilized single stranded nucleic acids, wherein said nucleic acid is being hybridised to RNA-protein fusions.

U.S. Pat. No. 6,194,550 describes a method called SPERT (Systemic Polypeptide Evolution by Reverse Translation), where a single stranded nucleic acid is immobilised on a solid support. A ribosomal complex is added and the protein corresponding to the immobilised gene sequence is then synthesized. The ribosome complex is removed and the solid support contains an immobilized nucleic acid-protein complex.

The present invention differs from prior approaches in that the proteins/peptides encoded by any nucleic acid sequence in the arrays are present and presented without the extra steps of transcription and translation of the cDNA on the array, since the present invention creates a protein display array as if the proteins had been formed by transcription and translation of the cDNA in the original library array. But instead the transcription and translation of the cDNA occurred during the propagation of the phage library.

Current protein array assemblies involve automation by mechanical and chemical deposition of proteins in array formats in (de Wildt et al. 2000, MacBeath and Schreiber 2000). In these cases the proteins may become disrupted or changed when bound to the solid array surface. The hybridisation step according to the present invention avoids this problem by leaving the protein/peptide displaying virion scaffold in solution.

### SUMMARY OF THE INVENTION

The present invention relates to a display virus and methods for producing coded nucleic acid arrays which display a diverse population of peptides or protein. The nucleic acid coded protein or peptide display arrays described in the present invention can be used in functional genomics, proteomics and in protein or peptide identification of relevance for the exploration of therapeutic drugs as well as for search of new diagnostic procedures. The present invention also relates to a kit comprising the display virus complex.

The nucleic acid coded protein or peptide display array technology described here has the important features of self-assembly and auto-addressing a protein product to its gene while keeping the displayed protein peptide in solution. The array can be reused after removal of the hybridised DNA-virion-fusion complexes by nucleic acid denaturation.

### BRIEF DESCRIPTION OF THE FIGURES

FIG. 1 A provides an electronic microscope picture of a phage P4 naked DNA-virion protein complex.

FIG. 1B depicts electron microscope picture of Lambda tail::DNA complexes.

FIG. 1C provides a drawing of ss-DNA tail complex displaying a protein.

FIG. 2 depicts a drawing of a magnification of one display virion unit in the process of being deposited by hybridisation in a DNA-array after DNA:DNA hybridisation in the array. The bottom drawing shows a virion-DNA display complex hybridising to a DNA-Array entry.

FIG. 3 depicts Lambda chromosome release from the particle and its simultaneous cross-linking to the tail/head protein components. An equal amount of Lambda particles in NaCl-EDTA buffer 0.02M NaCl, 0.005M Na₂EDTA (pH 7.4)) were disrupted by addition of high-pH buffer (pH 9) (0.068M Na₂CO₃, 0.0107 M Na₂EDTA, Chattoraj and Inmann, 1974) for two hours in the absence (lane 2) and presence of 10% HCHO (lane 3) and subjected to gel electrophoresis (0.7%) for two hours. The same amount of untreated particles are present in lane 1 (poorly stained) Lambda DNA in lane 4 serves as a reference.

FIG. 4 provides an asymmetric release of Lambda DNA chromosomes from Lambda phage particles

FIG. 4 A provides a drawing of KpnI treated Lambda DNA::tail/head complexes.

FIG. 4 B and C provides an agarose gel of KpnI treated-Lambda DNA::tail/head complexes, wherein 4C depicts the same agarose gel ran for 5 hours longer. The presence of different HCHO concentrations are shown.

FIG. 5 depicts autoaddressing Lambda protein display in a DNA array format

Arrays of alternating heat denatured Lambda DNA (●) and ØX174 RF-DNA (◯) (0.5 ug) hybridized with:
A. Lambda protein-naked DNA complexes untreated with exo-III nuclease as probe.
B. Lambda protein-naked DNA complexes treated with Lambda exo-III nuclease as probe.
C. In the absence of Lambda protein-naked DNA complexes (no probe).

Lambda and ØX174 DNA in the array=0.5 ug/spot, Lambda::DNA probe corresponding to 10⁹ particles Exo-III treatment for 2 hours. Hybridisation over night at 65 degrees

FIG. 6 depicts display of Lambda proteins in a DNA array format.

FIG. 7 depicts display of Lambda proteins in a DNA array format as a function of probe concentration.

FIG. 8 provides a covalent linkage plan of naked DNA display complex in a synthetic oligo nucleotide array.

### DEFINITIONS

As used herein, the term “virion” refers to virus particles exemplified by bacteriophages

As used herein, the term “naked DNA” or “naked nucleic acid” refers to the free bacteriophage DNA released from the virion. However, it could still comprise proteins and/or other biomolecules.

As used herein, the term “naked DNA tail/head complexes” refers to the released and naked bacteriophage DNA attached to the virion proteins making up the “tail” and the “head”.

As used herein, the term “naked nucleic acid virion protein display” denotes the naked DNA tail/head complexes displaying a peptide or protein.

As used herein the term “virion::protein” is defined as virion displaying a peptide or protein.

As used herein the term DNA virion fusion complexes refers to the naked DNA tai/head complexes displaying a peptide or protein

As used herein the term “cis-capture” is defined as the linking of peptide/protein or a protein complex to the very same template that encoded these protein entities.

### DETAILED DESCRIPTION OF THE INVENTION

The present invention relates to a display virus and methods for producing naked nucleic acid arrays which display a diverse population of peptides or protein, where said naked nucleic acid comprises exogenous nucleotide sequence(s) coding the protein/peptide. The nucleic acid coded protein or peptide display arrays described in the present invention can be used in functional genomics, proteomics and in protein or peptide identification of relevance for the exploration of therapeutic drugs as well as for search of new diagnostic procedures.

The present invention also relates to a kit comprising the display virus complex

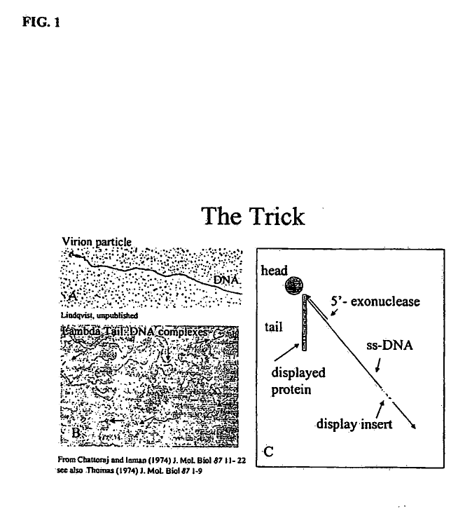

The present invention describes methods for protein/peptide display in nucleic acid arrays by integrating phage display in array formats e.g. a chip array format. The method takes advantage of a published observation (Chattoraj and Inman, 1974) that after specific treatments, more than 95% of the chromosomes of certain phages (e.g. Lambda, 186, P2 and P4) can be released naked from the phage capsids while remaining covalently attached—by one of their ends (always the same end)—to the top of their respective tail sometimes associated with the head (here referred to as a naked nucleic acid-virion protein complex and exemplified by phage P4 (Lindqvist unpublished observation, FIG. 1). This observation makes it possible to generate naked nucleic acid-virion protein display complexes in which the individual nucleic acid template is freely and covalently linked to the very same virion proteins it coded for. This cis-capture of the virion proteins by its naked nucleic acid template makes it possible to use the converted phage display libraries in combinatorial display array formats. This can be achieved by allowing the naked nucleic acid-virion protein display complexes to be deposited by nucleic acid hybridisation to their corresponding mRNA or cDNA which separately have been prepared and used for the fabrication of a nucleic acid array. The hybridisation step will therefore function as a ‘search engine’ and a ‘delivery robot’ by automatically positioning the protein to its own gene (sometimes related gene due to cross hybridisation) in the array by gently depositing the virion protein display complex to the solid surface. This procedure thus creates a protein display array as if the proteins had been formed by transcription and translation of the cDNA in the array. Instead the transcription and translation of the cDNA occured during the propagation of the phage library in E. coli.

In addition to arrays deposited two-dimensionally on e.g chips, the technology can also be used to deposit proteins in three dimensional arrays on particles in solution. Alternatively, the screening of ligands to the various proteins displayed on the virion could occur in solution, followed by capture of the complex by hybridisation of DNA strand in the naked nucleic acid virion protein display to the complementary DNA strand on either a DNA chip or on coated beads. Such solution-based interaction will enhance the speed and easy with which such interaction could occur.

Creating a Nucleic Acid Based Protein Display Array

In a preferred embodiment Lambda is applied since Lambda can display functional proteins fused both to its tail or head, by utilizing the V or the D proteins respectively (Maruyama et al. 1994, Dunn, 1995, 1996, Sternberg and Hoess 1995, Mikawa et al. 1996). V consists of 246 amino acids and it is present as 192 copies in the form of 32 hexameric discs. Since the most efficient chromosome cis-capture occurs with the tail under the present protocols, Lambda particles displaying a library of peptides or cDNA encoded proteins fused to the C-terminal part of the major tail protein V are used for the conversion into naked nucleic acid-virion protein display complexes. Certain Lambda mutants defective in termination of tail assembly form polytails, if functional in the assembly of particles, such polytails may be used to enhance the display capacity of phage array display.

A protein display array can then be created, where samples of individual phage stocks are used or individual plaques originating from a phage display library are picked and then treated to yield pure naked DNA-tail/head display complexes followed by their deposition in an array format by specifically cross-linking the DNA part to a solid support. The tail will then function as a giant free moving scaffold in which peptides or proteins fused to the V protomers are displayed. The steps of array assembly should be amendable to automation in a similar way described by de Wildt et al. (2000) or MacBeath and Schreiber (2000). The nature of any protein in the array interacting with a defined target or displaying a measurable function can easily be deduced by sequencing its stored phage replica, a procedure which would not differ from that used in standard phage display.

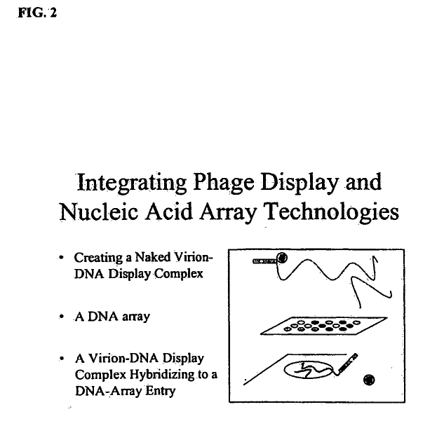

Another preferred embodiment is to hybridise the naked DNA-tail/head display complexes against a complementary cDNA library array or a synthetic oligonucleotide array, in such a way that each member of the peptide/protein library will hybridise to its homologue cDNA sequence if present in the array (FIG. 2). In order to avoid cross-hybridisation the cDNA present in the array should be a part of a vector other than Lambda Before hybridisation, however, the DNA bound to the tail/head has to be made single stranded by exonucleases such as either exoIII (3′) or Lambda exo (5′) degradation, one of the ends of the chromosome is hidden in the tail which protects it from exo-nuclease attack. This treatment generates ssDNA-tail/head display complexes as well as removes free DNA which failed to cis-capture the tail/head display complex. Since the phage library is amplified after its construction, a variable number of copies (the size of which will depend on the size of the library and the relative frequency of the individual mRNA/cDNA molecules converted to display format) of each unique DNA-tail/head display complex should be present and accessible for hybridisation. Again, the DNA-DNA hybridisation step will therefore function as a ‘search engine’ and a ‘delivery robot’ by automatically positioning the protein to its own (or related) gene in the array by gently depositing the tail/head display complex to the solid surface. This procedure thus creates a protein display array as if the proteins had been formed by transcription and translation of the cDNA in the original library array. But instead the transcription and translation (assuming an equal and constant translation of the cDNA during the phage propagation, the protein deposited in each position of the array should reflect the relative frequency of mRNA molecules in the cell at the time of harvest and not necessarily the protein amount) of the cDNA occured during the propagation of the phage library.

The efficiency of display in the array will be a function of the ratio between the size of the library and the number of cDNA clones in the array. If an array consists of 1000 cDNA clones originating from an organism of approximately 5000 genes the DNA-tail/head display library must carry a cDNA representative of each of those genes in order to be fully “expressed”. The Lambda V display vectors should easily handle up to 10⁷ independent clones and a 5×10³ cDNA library can be made to consist of up to 10⁸ copies or more of each cDNA clone after amplification. Therefore, each cDNA spot in the array can potentially contain up to 10⁸ or more copies of its encoded protein (assuming that one to two protein molecule are expressed as a fusion of V per particle). This is known to be the case for functional proteins expressed as fusions of V whereas peptides can be expressed as fusions of each V protomer (Maruyama et al 1994, Dunn, 1995, 1996) thereby increasing the presentation by a factor by 192 in each spot. In either case the amount of displayed peptide/protein in each spot will fall in the pg and sub-pmole range.

The present invention comprises a display virus complex exposing a naked nucleic acid comprising exogenous nucleic acid and its encoded peptide or polypeptide. The invention also relates to a a method of preparing covalently linked naked nucleic acid-protein display complexes from said virus particles, comprising at least the steps of:
a) treating a freshly prepared virus preparation with cross linking chemical agents producing covalently linked naked nucleic acid-virus protein display complexes,
b) coupling of the naked nucleic acid-virus protein display complexes to a solid support, by hybridising of the naked nucleic acid-virus protein display complexes against a complementary nucleic acid sequence in a array format and where said hybridisation leads to positioning the displayed protein/peptide to its own gene or related gene(s). Said coupling in b) can also be is performed by chemical cross linking agents to a solid support. The present invention relates further to use of the method and a kit comprising the virus display complex.
   Use of the Method

The nucleic acid coded protein or peptide display arrays described here can be used in functional genomics, proteomics and in protein or peptide identification of relevance for the exploration of therapeutic drugs as well as in the search for new diagnostic procedures. The array can be reused after removal of the hybridised DNA-virion-fusion complexes by nucleic acid denaturation. The naked nucleic acid-virion protein display complex can also be used as a scaffold for bi-functional display after nucleic acid hybridisation of two different display complexes.

Results

Cis-Capture of the Phage Chromsome to its Tail/Head Components.

Of the phages known to be amenable to chromsome release and cis-capature by the tail/capsid components, only phage Lambda has been developed for peptide as well as c-DNA display (Lindqvist, unpublished). P4 has only been shown to function in the presentation of a c-myc peptide by the capsid component Psu as a display module.

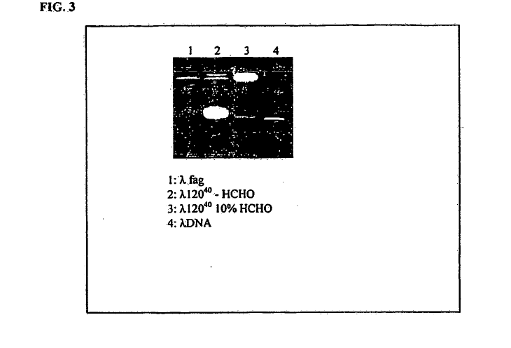

To achieve chromosome release and its covalent cis-capture to the tail/head components, Lambda particles—typically 2×10¹¹ in NaCl-EDTA buffer were mixed with high-pH buffer (pH 9) as described by Chattoraj and Inman (1974) and kept for two hour at 40 degrees in the presence of the cross-linking agent HCHO (10% final concentration). After agarose gel electrophoresis of the samples (FIG. 3), the vast majority of the DNA of the HCHO treated particles remain in the well as DNA::tail/head complexes (lane 3) while the DNA of the HCHO untreated particles migrate as a free Lambda DNA band (lane 2) as compared to a Lambda DNA marker (lane 4). Intact Lambda phage particles remain in the well and only weakly respond to the ethidium bromide staining (lane 1). These results are consistent with the observations of Chattoraj and Inman (1974) that HCHO treatment leads to covalent cis-capture of the Lambda chromosome to the tail/head components. For further use the complexes are dialysed against water.

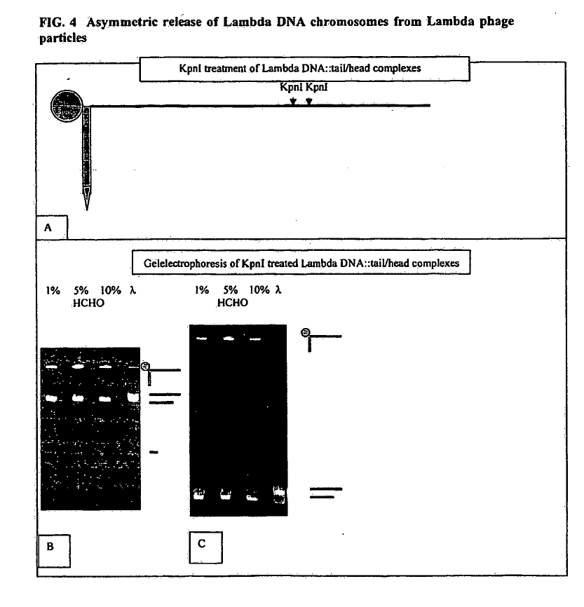

To demonstrate the presence of released DNA in the HCHO generated DNA::tail/head complexes and to monitor the orientation of the chromosome release, restriction enzyme KpnI was used to cut the naked Lambda DNA as outlined in FIG. 4A. Prior to cutting the DNA::tail/head complexes were dialysed against water for 30 minutes.

After agarose gel electrophoreses of samples treated with 1, 5 and 10% HCHO respectively two bands could be seen migrating in 4 B while roughly half of the material was retained in the wells. The fastest moving band corresponds to the small central KpnI fragment. This band disappeared after further electrophoreses while the slow moving band in 4 B was split into two bands in 5 C (KpnI treated Lambda DNA control, right). In all three cases (1, 5 and 10% HCHO treatment) the fastest moving band in 4 C was the most abundant demonstrating the directed release of the Lambda chromosome as outlined in 4 A and originally reported by (Chattoraj and Inman 1974 and Thomas 1974).

Hybridisation of the Lambda DNA::Tail/Head Complexes in a Lambda DNA Array

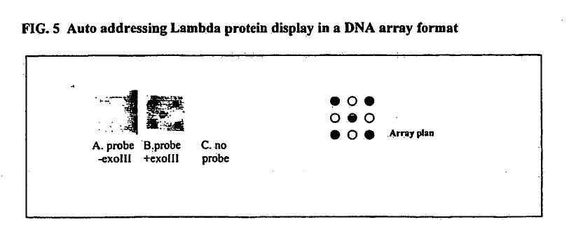

The DNA array is prepared by addition of alternating ss-DNA of Lambda and ØX174 as described in FIG. 5. Heat denatured Lambda or ØX174 DNA were spotted in an alternating pattern on a nitrocellulose filter and baked by heat-treatment at 80 degrees for one hour. Roughly 10⁹ of Lambda::naked DNA display complexes were added to a total volume of two and a half ml hybridisation solution and incubated in a hybridisation owen at 65 degrees over night. The DNA part of the Lambda DNA::tail/head complex was made single-stranded by exo-III treatment for two hour at 37 degrees prior to hybridisation. Display of Lambda proteins in the array was monitored by phage Lambda poly-clonal antibodies produced in a rabbit. Rabbit specific anti-G antibodies conjugated with horse-radish peroxidase was used as a secondary antibody in combination with the ECL visualization system of Amersham Biotech. For each step (hybridisation and protein detection) blocking of the filter was necessary. The ØX174 DNA serves as a control of the hybridisation specificity and addressable array format. In FIG. 5 the array filter A and B were thereby provides a demonstration of Lambda protein display in an auto-subjected to hybridisation with roughly 10⁹ DNA::tail/head complexes each. The only exception being that the probe present in A was not treated with exo-III. Hence neither hybridisation nor Lambda protein display is to be expected in A. In B, however, the Lambda DNA was made single-stranded by exo-III treatment. If the exo-III treatment works hybridisation and display of Lambda tail/head proteins should occur. No Lambda::tail/head probe was added to filter C that functions as a background control.

The result shown in FIG. 5 demonstrates clearly: 1. an acceptable background level in 4 C, 2. The auto-addressing of Lambda proteins to the Lambda DNA spots in the 5 B array and 3. an absolute requirement for ss-DNA for this to occur (compare 5 A and 5 B) (this result also demonstrates the effectivness of exo-III treatment in providing ss-DNA).

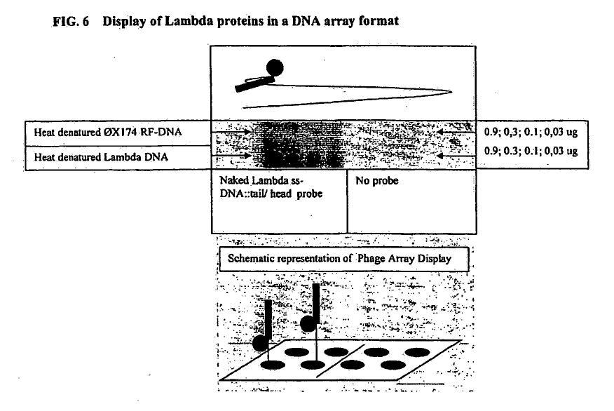

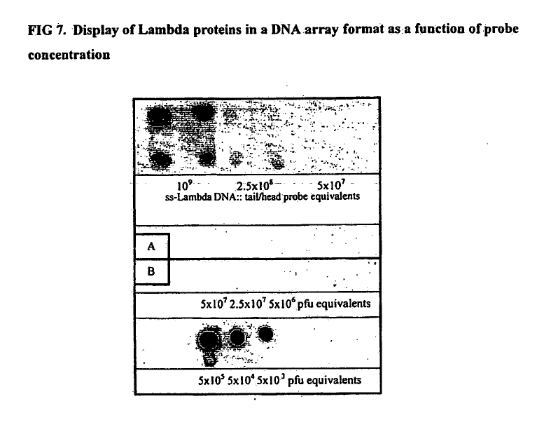

In FIG. 6 a nitro-cellulose filter with 0.9 to 0.03 ug of Lambda and ØX174 DNA respectively was subjected to ss-Lambda DNA::tail/head probe hybridisation. As can be seen a concentration dependent Lambda specific DNA hybridisation has occurred. Hence—a limiting amount of 0.03 ug Lambda DNA (or perhaps less) is required in the spot to detect display of Lambda protein under these conditions. In FIG. 7 A, 0.5 ug Lambda DNA in each spot is hybridized with 10⁹, 2.5×10⁸ and 5×10⁷ Lambda DNA::tail/head complexes respectively. In FIG. 7 B, different amounts of ss-Lambda DNA::tail/head probe were spotted on a separate nitrocellulose filter to create a standard curve (the DNA::tail/head complexes in the spots correspond to 5×10⁷, 2.5×10⁷, 5×10⁶, 5×10⁵, 5×10⁴ 5×10³ pfu equivalents respectively) and the Lambda proteins were detected simultaneous with those deposited after hybridisation (FIG. 7 A). By comparing the intensities of the spots it can be estimated that the efficiency of hybridisation—under current conditions—corresponds to in the order of 0.1%. This efficiency should be possible to improve to 10% by using ten times more probe equivalents and by decreasing the hybridisation volume to 250 μl.

Discussion

The Technology Platform

The technology platform described here relates to peptide and protein display in nucleic acid array formats. By a simple trick virion displayed peptides or proteins can be integrated and presented in a nucleic acid array format. This is done by generating naked nucleic-acid::virion protein display-complexes in which the individual nucleic acid template is freely and covalently linked to the very same virion proteins it coded for. This cis-capture of the virion proteins by its naked nucleic acid template will make it possible to use phage display libraries in combinatorial display array formats. The naked nucleic acid part of the virion protein display complexes is deposited in an array format by hybridisation to their corresponding mRNA/cDNA/genomic DNA that separately had been prepared and used in the array fabrication. The hybridisation step will therefore function as a ‘search engine’ and a ‘delivery robot’ by automatically positioning the protein to its own gene—as if the gene had been translated in the array instead of during the lytic propagation of the phage. The virion proteins (tail/head) will remain in solution anchored by nucleic acid complementarity and function as a display-scaffold for the particular peptide or protein. The amount of display is estimated to be in the pico- to nanogram range.

On the Formation of the Linkage Between the Display Complex and the Array Spot as a Result of DNA-DNA Hybridisation

The strength by which the display complex is linked in the array should reflect the complementary sequence length of the DNA engaged in hybridisation.

1) Poor processivity of the exonuclease during the single stranded DNA formation will lead to a reduced hybridisation capacity and linkage formation due to partial single-strandeness. This can be avoided by cutting the naked DNA::tail display complexes with a restriction enzyme next to the DNA sequence that is engaged in hybridisation before the exo-nuclease treatment. Furhtermore, by performing a strand elongation synthesis (in parallel with the hybridisation step) using the hybridising strand as a primer the number of hydrogen bonds can be increased leading to a tighter linkage between the display entity (the tail) and the array spot.

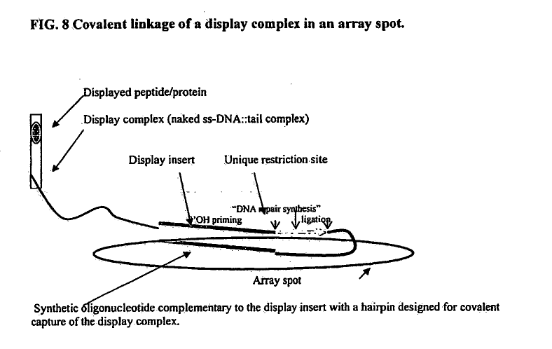

2) When only small sequence stretches are available such as during peptide display in conjuncture with synthetic oligo nucleotide based arrays, strand elongation synthesis will be limited or impossible to perform. In this case the link can be made covalent during hybridisation by simultaneously performing a “DNA repair” synthesis and a ligation reaction to a specially designed hairpin containing complementary oligo nucleotide as outlined in the FIG. 8. In the presence of dNTP, thermophilic DNA polymerase as well as ligase the tail containing hybridising strand will act as a primer for DNA synthesis by strand elongations (3′-5′) using the specially designed hairpin containing oligonucleotide in the array as a template and the ligase will covalently link the display complex to the oligo nucleotide. In this case a unique restriction site should follow the display insert in order to allow priming from a perfect base pair match. Under these conditions an exo-5′ nuclease has to be used instead of exoIII for single strand DNA formation in order to provide a 3′OH group for priming the DNA synthesis.

Technology Attributes—Attractiveness.

The technology integrates phage display with array technology and thus extends the use of the established phage display technology. The uniqueness of the proposed technology is the integration of phage display and array technologies for protein presentation (proteomics) and functional genomics. Traditional phage display is expanding exponentially and is now an established technology, widely known and dispersed in academic research and in industry. Hence, the array display described here should be an attractive alternative to co-existing technologies that already are protected by patents.

There are currently only two other technologies providing in solution display of peptides or proteins with a potential for self-assembly in array format. The Profusion™/Hip-Chip™ technology (Phylos Inc, Lexington, Mass., USA) and the CDT technology (Isogenica Ltd, Cambridge, UK, www.isogenica.com) represents two such technology platform. The PROfusion™ technology, for example, allows the production of nucleic acid-protein display-complexes that potentially can be integrated in array formats by their Hip™ chip technology.

In summary the present invention establishes a novel soft and simple way (nucleic acid hybridisation) of assembling protein arrays. But most importantly, the concept also provides a unique way to link proteins to their genes by peptide or protein display in an array format. Hence, the present invention emerges as a technology platform for functional genomics, proteomics that relates to drug exploration—in constant demand by the pharmaceutical industry in their quest for novel drug candidates. Furthermore array formats like PAD can be developed into automated multi-channel diagnostic systems. The technology platform may also have a potential for use in combinatorial self-assembling processes and thus have implications for nano-science and the budding field of nano-technology.

### REFERENCES

1. Cahill, D. Protein arrays: a high-throughput-solution for proteonomics research. Proteonomics: A trends Guide. (2000) July, 47-51
2. Chattoraj and Inman. Location of DNA ends in P2, 186, P4 and Lambda bacteriophage heads. J. Mol. Biol. (1974) 87, 11-22
3. Davis, R. W., Simon, M. and N. Davidson. In Methods in Enzymology (1971) 21, part D 414-428
4. DeWildt, R. M., Mundy, C. R., Gorick, B. D. and I. M Tomlinson. Antibody arrays for high-throuput screening of antibody-antigen interactions. Nat. Biotechnology (2000) 18 989-994
5. Dunn I. S. Assembly of functional bacteriophage Lambda virions incorporating C-terminal peptide or protein fusion with the major tial protein. J. Mol. Biol (1995) 248, 497-506
6. Dunn I. S. Total modification of the bacteriophage Lambda tail tube major subunit protein with foreign peptides. Gene (1996) 183, 15-21
7. Irving, R. A. and P. Hudson. Proteins emerge from disaaray. Nat. Biotchnology (2000) 18, 932-933
8. Lindqvist, B. H. and S. Naderi. Peptide presentation by bacteriophage P4. FEMS Microbiol. Rev. ((1995) 17, 33-39
9. MacBeath, G and S. L. Schreiber. Printing Proteins as Microarrays for High-throughput Function Determination, Science (2000) 289, 1760-1763
10. Maruyama I N, Maruyama H I, S. Brenner. Lambda foo: a Lambda phage vector for the expression of foreign proteins. Proc Natl Acad Sci USA. (1994) Aug. 16; 91 (17):8273-7.
11. Mikawa Y G., Maruyama, I. N. and S. Brenner. Surface display of proteins on bacteriophage Lambda heads. J. Mol Biol (1996) 262, 21-30.
12. Mitchell, P. A perspective on protein microarrays. Nature Biotechnol. (2002) 20, 225-229
13. Roberts, R W, Szostak J W. RNA peptide fusions for the in vitro selection of peptides and proteins. Proc Natl Acad Sci USA, 1997 Nov. 11; 94 (23):12297-302
14. Sternberg, N and R. H. Hoess. Display of peptides and proteins on the surface of bacteriophage Lambda. Proc. Nat. Acad. Sci (USA) (1995) 28, 1609-1613
15. Thomas, J. O. Chemical linkage of the tail to the right-hand end of bacteriophage Lambda DNA. J. Mol. Biol. (1974) 87, 1-9

What is claimed is:

1. A display virus complex exposing a naked nucleic acid comprising exogenous nucleic acid and its encoded peptide or polypeptide.

2. The display virus complex of claim 1, wherein said naked nucleic acid is covalent linked to the protein it coded for.

3. The display virus complex of claim 1, wherein the said naked nucleic acid-virion protein display complexes are fabricated from head and tail containing virus particles by covalent cis-capture of the virion by the naked nucleic acid chromosome.

4. The display virus complex of claim 1, wherein a purified stock/plaque of virus particles displaying the protein/peptide is the source of said naked nucleic acid-virion protein display complexes.

5. The display virus complex of claim 1, wherein viral display libraries of proteins/peptides are the source of said naked nucleic acid-virion protein display complexes.

6. The display virus complex of claim 1, wherein the displayed protein/peptide of the naked nucleic acid-virion protein display complex is a functional or a nonfunctional protein or a peptide

7. The display virus complex of claim 1, wherein said naked nucleic acid comprises the nucleotide sequence coding the protein/peptide of claim 6.

8. The display virus complex of claim 1, wherein said virus is a bacteriophage particle.

9. The display virus complex of claim 8, wherein the bacteriophage particle is Lambda.

10. The virus of claim 1, wherein said nucleic acid is a DNA or a RNA molecule.

11. The virus of claim 10, wherein the DNA or RNA are single stranded.

12. A method of preparing covalently linked naked nucleic acid-protein display complexes from virus particles of claim 1, comprising at least the steps of:
a) treating a freshly prepared virus preparation with cross linking chemical agents producing covalently linked naked nucleic acid-virus protein display complexes,
b) coupling of the naked nucleic acid-virus protein display complexes to a solid support, by hybridising of the naked nucleic acid-virus protein display complexes against a complementary nucleic acid sequence in a array format and where said hybridisation leads to positioning the displayed protein/peptide to its own gene or related gene(s).

13. The method of claim 12 wherein said coupling in step b) is performed by chemical cross linking agents to a solid support.

14. The method of claim 12, wherein prior to the hybridization in step b) the nucleic acid of the virus complex is cut with a restriction enzyme next to the DNA sequence that is engaged in the hybridisation before making the nucleic acid single stranded.

15. The method of claim 14, wherein a strand elongation synthesis is performed in parallel with the hybridisation step by using the hybridising strand as a primer.

16. The method of claim 14, wherein a DNA repair synthesis and a ligation reaction is performed simultaneously to the hybridisation step by using a specially designed hairpin comprising complementary oligo nucleotides.

17. The method of claim 12, wherein the said naked nucleic acid-virus protein display complexes are fabricated from head and tail containing virus particles by covalent cis-capture of the virion by the naked nucleic acid chromosome.

18. The method of claim 12, wherein a purified stock/plaque of virus particles displaying the protein/peptide is the source of said naked nucleic acid-virion protein display complexes.

19. The method of claim 12, wherein viral display libraries of proteins/peptides are the source of said naked nucleic acid-virion protein display complexes.

20. The method of claim 12 wherein the displayed protein/peptide of the naked nucleic acid-virion protein display complex is a functional or a nonfunctional protein or a peptide.

21. The method of claim 12 wherein said naked nucleic acid comprises the nucleotide sequence coding the protein/peptide of claim 20.

22. The method of claim 12 wherein said solid support is in an array format.

23. The method of claim 12 wherein the naked nucleic acid-virion protein complexes are kept in a solution for hybridisation purposes.

24. The method of claim 12 wherein the bi-functional naked nucleic acid-virion protein display complexes can be formed by hybridisation.

25. Use of the method of claim 12 in functional genomics, proteomics and in protein or peptide identification for the exploration of therapeutic drugs as well as in the search for new diagnostic procedures.
